(19) United States
(12) Patent Application Publication
(10) Pub. No.: US 2026/0335244 A1
(43) Pub. Date: Oct. 29, 2026

(54) USE OF CARBOHYDRATE-CONTAINING COMPOSITIONS IN TABLET FORM FOR TREATMENT OF HYPO-GLYCEMIC STATES

(71) Applicant: EUVORIO GMBH, Köln (DE)

(72) Inventor: Julius GRENNIGLOH, Köln (DE)

(21) Appl. No.: 19/491,157

(22) PCT Filed: Jun. 10, 2024
(86) PCT No.: PCT/EP2024/065870

§ 371 Date: Dec. 8, 2025

(30) Foreign Application Priority Data
Jun. 9, 2023 (DE) 10 2023 002 336.1

(51) Int. Cl. A61K 9/28; A61K 9/20; A61K 31/7004; A61K 31/716; A61K 31/718

(52) U.S. Cl. CPC A61K 9/2866; A61K 9/2068; A61K 9/284; A61K 31/7004; A61K 31/716; A61K 31/718

(57) ABSTRACT

Special carbohydrate-containing compositions and tablet forms thereof configured for the preparation of agents for the treatment of hypoglycemia, in particular nocturnal hypoglycemia. The compositions and tablet forms are easy to dose and do not leave a sweet taste in the mouth, but quickly release the carbohydrates they contain in the stomach and are therefore particularly suitable for treating nocturnal hypoglycemia.

This application is a 371 of International Patent Application No. PCT/EP2024/065870, filed Jun. 10, 2024, which claims foreign priority benefit under 35 U.S.C. § 119 of the German Patent Application No. 10 2023 002 336.1, filed Jun. 9, 2023, the disclosures of which are incorporated herein by reference.

The invention relates to the use of special carbohydrate-containing compositions, in particular in tablet form, for the preparation of agents for the treatment of forms of hypoglycemia, in particular nocturnal hypoglycemia.

Diabetes mellitus is a metabolic disorder that affects the utilization of carbohydrates. In particular, it is caused by a lack of insulin and can lead to chronic hyperglycemia with numerous consequences.

In the body's digestive system, the carbohydrates ingested with food are broken down into glucose, which is transferred into the blood through the intestinal wall and distributed throughout the body. The body's own hormone insulin causes the glucose contained in the blood to enter the cells, where it is used to produce energy. The insulin required for this is mainly produced in the pancreas. If the body does not produce any or sufficient insulin, glucose cannot be transferred into the body's cells and therefore remains in the blood. To avoid the consequences of high blood sugar levels, diabetes mellitus is usually treated with insulin. This poses the problem of correctly dosing the amount of insulin, i.e. adjusting it to the amount of carbohydrates. If too little insulin is given, too much glucose remains in the blood. If the insulin dosage is too high, this can lead to low blood sugar (hypoglycemia). Hypoglycemia of this kind is a serious disorder that can be fatal in the worst case.

People who suffer from diabetes therefore often carry carbohydrate-containing foods with them in order to be able to quickly effect a carbohydrate intake in the event of low blood sugar. Commonly used remedies are glucose tablets, fruit juices or bread.

Hypoglycemia can also occur at night. In the event of low blood sugar, people suffering from diabetes wake up at night sweating, for example, or are woken up by warning devices. This can occur, for example, if the person concerned ate a meal in the evening or at night whose exact carbohydrate content is not known. The person then has to estimate the amount of carbohydrates in order to inject the appropriate amount of insulin. If the amount of carbohydrates is overestimated, this can lead to the aforementioned low blood sugar after insulin administration.

Persons concerned often find it uncomfortable to eat glucose, (sweet) fruit juices or bread, especially at night, to compensate for the low blood sugar. To compensate for the sweet taste in their mouths, they would have to get up at night and brush their teeth again, for example. Therefore, the object is to provide a composition that is able to raise blood sugar levels in a targeted manner in the event of low blood sugar, is easy to dose and at the same time does not leave an unpleasantly sweet taste in the mouth, so that persons concerned can continue to sleep. This object is solved by the use of the compositions described below.

The following publications are known from the prior art:

Document US2014/0180224 A1 describes compositions for the treatment of nocturnal hypoglycemia. However, these are compositions that are described as delayed and sustained release compositions. The compositions should be taken before going to bed and ensure a steady supply of carbohydrates during the night. Treatment of acute nocturnal hypoglycemia is not possible due to the delayed release.

The document CN 115226896 describes further sustained release compositions of various carbohydrates, which are also not suitable for the treatment of acute nocturnal hypoglycemia.

The document DE 2246013 A describes a method for the preparation of porous carbohydrate-containing tablets, which are intended to solve various preparation problems of tablet formulations. Nocturnal hypoglycemia treatment is not mentioned here.

Document WO 2015148742 A1 describes compositions for the preparation of cell cultures. These compositions are intended to contain carbohydrates and are described as sustained release compositions. Use for nocturnal hypoglycemia treatment is not mentioned here

The document CN 105963266 A describes glucose tablets that may also be intended to be suitable for the treatment of hypoglycemia. Due to their composition, the tablets dissolve in the mouth and produce a sweet flavor.

The present invention relates to the use of carbohydrate-containing compositions, in particular in tablet form, which release the contained carbohydrates only in the gastrointestinal tract, for the treatment of hypoglycemia, in particular nocturnal hypoglycemia.

These compositions initially contain one or more saccharides as carbohydrates, such as dextrose, lactose, maltose, trehalose, sucrose or mixtures thereof. A well-known carbohydrate mixture is maltodextrin, for example. Some of the carbohydrates occur in different hydrate forms, i.e. they contain different amounts of water of crystallization. Dextrose is available as a monohydrate, for example, but also in an anhydrous form. Typically, all available stable hydrate forms can be used for preparing the preparations according to the invention.

The advantage of dextrose is that it acts quickly, while other carbohydrates have a delayed onset of action, but may have a longer-lasting effect. Mixtures of different carbohydrates can produce a rapid initial effect, which lasts longer when more complex carbohydrates are added.

For the purpose mentioned at the beginning, the composition consists predominantly, i.e. more than 70%, of the saccharide or saccharide mixture. Only agents customary in the art for improving processability and dissolution properties, respectively, are added, such as stearates or cellulose. The saccharide content is up to 95%, typically around 80%. The most preferred saccharide according to the invention is dextrose, in particular as dextrose monohydrate.

First, the composition is pressed into pellet-shaped tablet cores. The individual tablet cores have diameters of between 4 and 10 mm, preferably between 6 and 8 mm, and may have the usual shapes for such compositions, such as spherical, biconvex, faceted or domed. It should be understood that for the purpose of ingestion, they should not have any sharp edges, otherwise the shape is virtually unlimited.

According to the invention, the prefabricated tablet cores are coated with a coating that is neutral in taste and ensures that the tablets do not dissolve in the mouth, but that the saccharides they contain are only released in the stomach. Further, the coating facilitates swallowing the tablets. Such coatings are known in principle. These are usually composed of starch and modified cellulose. The decisive factor for the coating is that it does not yet dissolve in the mouth, but only quickly releases the tablet core in the stomach. Preferably, the following substance classes are used for the coating according to the invention:
Cellulose and cellulose derivatives, in particular carboxymethyl cellulose, hydroxypropyl methyl cellulose phthalate, methyl cellulose, hydroxypropyl methyl cellulose (hypromellose) and/or hydroxypropyl cellulose
Methacrylic acid copolymers and methacrylic acid esters
Polyvinyl pyrrolidone (PVP) and derivatives
Polyvidone acetate
Polyvinyl acetate phthalate

The most preferred coating agent is hydroxypropyl methyl cellulose (hypromellose). The coatings may also contain colorants. Preferred food colorants used for this purpose are for example carotenoids (E 160a), berry colorants (anthocyanins, E 163), beets colorants (betanin, E 162), carmine (E 120), paprika extract (E 160c) and curcumin (E 100). Strongly coloring plant or fruit extracts such as beetroot, spinach or elderberry juice and spices such as saffron and turmeric are particularly preferred.

For practical reasons, the tablet cores are preferably coated with the coating in a single process, resulting in modified film-coated tablets (film-coated dragées). Alternatively, classic coating with several layers is also possible, of course.

The tablets formed from the tablet cores by coating typically have a diameter of between 4 and 10 mm and a mass of between 50 and 500 mg. Preferably, the individual tablets have a diameter of 7 to 8 mm and a mass of 210 to 270 mg.

A person suffering from low blood sugar can dose the composition according to the invention in tablet form using simple dosing devices, for example a dosing spoon. The spoon can be designed in such a way that a total amount of tablets is ingested that corresponds to a certain amount of carbohydrates, for example a carbohydrate unit, the typical calculation quantity for diabetics. One or more carbohydrate units can then be removed from a storage container and taken orally, as needed. A drink of water can be helpful to swallow the tablets completely. As already described above, the tablets do not release carbohydrates into the mouth within approx. 15 seconds, so that their sweet taste in the mouth is avoided. The tablets only dissolve in the stomach, where they quickly release the saccharides they contain, which then combat low blood sugar by raising the blood sugar level. Within one minute of swallowing, more than 80% of the carbohydrate content of the tablets are released in an empty stomach. This avoids the usual sweet taste of carbohydrate-containing compositions. At the same time, there is no need for brushing the teeth for the purpose of caries prevention after administration. In the event of low blood sugar at night, the person concerned can then go back to sleep immediately.

The properties of the tablets described also allow them to be used for hypoglycemic conditions that are not diabetes-related, e.g. in endurance sports. For example, marathon runners may experience what is known as “hitting the wall”, which in turn is a form of hypoglycemia that is not diabetes-related. Another form of non-diabetes-related hypoglycemia can occur when people have not eaten for a long time or have only eaten extremely low carbohydrate (“fasting induced hypoglycemia”). A dosed intake of the tablets described can also overcome such hypoglycemic conditions.

A particular advantage of the tablets described is that, if taken as described, a hypoglycemic condition can be treated without the risk of triggering sugar addiction. Persons skilled in the art believe that such sugar addiction is encouraged by the association of sweet taste with the immediate subsequent rise in blood sugar, as occurs when eating common sweets (e.g. chocolate bars, gummy bears). The absence of the sweet taste in the mouth apparently does not activate the reward system in the brain, so that no addictive effects occur.

### EXAMPLES

The following examples further explain the invention without wishing to be restrictive. The person skilled in the art can develop other embodiments of the invention based on the examples without having to be inventive.

### Example 1

### Tablet Core:

| Raw material | Content % |
|---|---|
| Dextrose monohydrate | 76.0 |
| Sodium croscarmellose | 8.2 |
| Microcrystalline cellulose | 15.8 |

For the preparation of the tablet cores, the components listed above are mixed and pressed into tablet cores. The coating is then applied with the composition specified below.

Coating: Biogrund AquaPolish F pink 040.344 or AquaPolish F pink 044.419

| Raw material | Quality |
|---|---|
| Hydroxypropyl methyl cellulose | E464 |
| Beetroot powder (optional) | Food ingredient |
| Macrogol | E1521 |
| Rice starch | Food ingredient |
| sucrose esters of fatty acids | E473 |
| Natural carmine | E120 |

After drying, the tablets can be packaged.

### Product Description:

Tablet size: 7 or 8 mm tablet cores (domed)
Tablet core mass: 238 mg
Tablet mass (with coating) 247 mg

### Example 2

### Tablet Core:

| Raw material | Content % |
|---|---|
| Trehalose dihydrate | 85.2 |
| Sodium croscarmellose | 5.2 |
| Microcrystalline cellulose | 9.6 |

For the preparation of the tablet cores, the components listed above are mixed and pressed into tablet cores. The coating is then applied with the composition specified below.

Coating: Biogrund AquaPolish F pink 040.344 or AquaPolish F pink 044.419

| Raw material | Quality |
|---|---|
| Hydroxypropyl methyl cellulose | E464 |
| Beetroot powder (optional) | Food ingredient |
| Macrogol | E1521 |
| Rice starch | Food ingredient |
| sucrose esters of fatty acids | E473 |
| Natural carmine | E120 |

After drying, the tablets can be packaged.

### Product Description:

Tablet size: 7 or 8 mm tablet cores (domed)
Tablet core mass: 238 mg
Tablet mass (with coating) 247 mg

### Example 3a

### Tablet Core:

| Raw material | Content % |
|---|---|
| Dextrose monohydrate | 81.8 |
| Sodium croscarmellose | 6.2 |
| Microcrystalline cellulose | 12.0 |

For the preparation of the tablet cores, the components listed above are mixed and pressed into tablet cores. The coating is then applied with the composition specified below.

Coating: Biogrund AquaPolish F pink 040.344 or AquaPolish F pink 044.419

| Raw material | Quality |
|---|---|
| Hydroxypropyl methyl cellulose | E464 |
| Beetroot powder (optional) | Food ingredient |
| Macrogol | E1521 |
| Rice starch | Food ingredient |
| sucrose esters of fatty acids | E473 |
| Natural carmine | E120 |

After drying, the tablets can be packaged.

### Product Description:

Tablet size: 7 or 8 mm tablet cores (domed)
Tablet core mass: 238 mg
Tablet mass (with coating) 247 mg

### Example 3b

### Dragée Core:

| Raw material | Content % |
|---|---|
| Dextrose monohydrate | 82.4 |
| Sodium croscarmellose | 6.2 |
| Cellulose | 11.4 |

For the preparation of the dragee cores, the components listed above are mixed and pressed into dragee cores. The coating is then applied with the composition specified below.

Coating: Biogrund Aquapolish F green 074.133 MS

| Raw material | Quality |
|---|---|
| Hypromellose | E464 |
| Macrogol | E1521 |
| Rice starch | Food ingredient |
| sucrose esters of fatty acids | E473 |
| Hydroxypropyl cellulose | E463 |
| Microcrystalline cellulose | E460(i) |
| Copper chlorophyllin | E141(ii) |

After drying, the dragées can be packaged.

### Product Description:

Dragée size: 7 or 8 mm dragée cores (domed)
Dragée core mass: 238 mg
Dragée mass (with 247 mg coating)

### Example 3c

### Dragée Core:

| Raw material | Content % |
|---|---|
| Dextrose monohydrate | 81.2 |
| Magnesium stearate | 1.2 |
| Sodium croscarmellose | 6.2 |
| Cellulose | 11.4 |

For the preparation of the dragee cores, the components listed above are mixed and pressed into dragee cores. The coating is then applied with the composition specified below.

Coating: Biogrund Aquapolish F green 074.133 MS or AquaPolish F pink 044.419

| Raw material | Quality |
|---|---|
| Hypromellose | E464 |
| Macrogol | E1521 |
| Rice starch | Food ingredient |
| sucrose esters of fatty acids | E473 |
| Hydroxypropyl cellulose | E463 |
| Microcrystalline cellulose | E460(i) |
| Copper chlorophyllin | E141(ii) |

After drying, the dragées can be packaged.

### Product Description:

Dragée size: 7 or 8 mm dragée cores (domed)
Dragée core mass: 238 mg
Dragée mass (with 247 mg coating)

### Example 4

### Tablet Core:

| Raw material | Content % |
|---|---|
| Dextrose monohydrate | 61.6 |
| Trehalose dihydrate | 21.8 |
| Sodium croscarmellose | 6.2 |
| Microcrystalline cellulose | 11.4 |

For the preparation of the tablet cores, the components listed above are mixed and pressed into tablet cores. The coating is then applied with the composition specified below.

Coating: Biogrund AquaPolish F pink 040.344

| Raw material | Quality |
|---|---|
| Hydroxypropyl methyl cellulose | E464 |
| Beetroot powder | Food ingredient |
| Macrogol | E1521 |
| Rice starch | Food ingredient |
| sucrose esters of fatty acids | E473 |
| Natural carmine | E120 |

After drying, the tablets can be packaged.

### Product Description:

Tablet size: 7 or 8 mm tablet cores (domed)
Tablet core mass: 238 mg
Tablet mass (with coating) 247 mg

### Example 5

### Tablet Core:

| Raw material | Content % |
|---|---|
| Dextrose monohydrate | 65.3 |
| Maltose hydrate | 16.5 |
| Sodium croscarmellose | 6.2 |
| Microcrystalline cellulose | 12.0 |

For the preparation of the tablet cores, the components listed above are mixed and pressed into tablet cores. The coating is then applied with the composition specified below.

Coating: Biogrund AquaPolish F pink 040.344

| Raw material | Quality |
|---|---|
| Hydroxypropyl methyl cellulose | E464 |
| Beetroot powder (optional) | Food ingredient |
| Macrogol | E1521 |
| Rice starch | Food ingredient |
| sucrose esters of fatty acids | E473 |
| Natural carmine | E120 |

After drying, the tablets can be packaged.

### Product Description:

Tablet size: 7 or 8 mm tablet cores (domed)
Tablet core mass: 238 mg
Tablet mass (with coating) 247 mg

### Example 6a

### Tablet Core:

| Raw material | Content % |
|---|---|
| Maltodextrin | 82.6 |
| Sodium croscarmellose | 6.2 |
| Microcrystalline cellulose | 12.0 |

For the preparation of the tablet cores, the components listed above are mixed and pressed into 6 mm diameter tablet cores.

### Example 6b

The tablet cores produced according to Example 6a will be coated with a coating, the coating having the composition given below.

Coating: Biogrund Aquapolish F green 074.133

| Raw material | Quality |
|---|---|
| Hypromellose | E464 |
| Macrogol | E1521 |
| Rice starch | Food ingredient |
| sucrose esters of fatty acids | E473 |
| Hydroxypropyl cellulose | E463 |
| Microcrystalline cellulose | E460(i) |
| Copper chlorophyllin | E141(ii) |

After drying, the tablets can be packaged.

### Product Description:

Tablet size: Approx. 7 mm tablet cores (domed)
Tablet core mass: 117 mg
Tablet mass (with coating) 124 mg

### Example 7: Sweet Presence Binary Test to Evaluate the Sweetness of Tablets

The question of whether a carbohydrate-containing composition produces a sweet taste in the mouth when used according to the invention can be tested by the Sweet Presence Binary Test described below.
Objective: Determine whether a tablet immediately tastes sweet after it has been in the mouth for more than 15 seconds.
Applicability: This protocol is applicable to all types of tablets that are intended for oral administration and should not be dissolved in the mouth but swallowed directly.

### Materials:

Test tablets (product to be tested)
Water to rinse the mouth between tests

### Test Subjects:

At least 20 test subjects (without sensory training), preferably approx. 100 people
Age between 18 and 65 years
No known taste impairments

### Method:

a. Preparation:
The test subjects are informed about the objective of the test and give their consent to participate.
Each test subject is given instructions not to eat any strongly flavored or sweet foods before the test.
   b. Implementation:
Each test subject receives a certain number of test tablets, corresponding to the usual dosage for the product being tested.
The test subjects hold the tablets in their mouths for 15 seconds before swallowing them.
Immediately after swallowing, the test subjects indicate whether or not they perceived any sweetness during the time in the mouth.
   c. Documentation:
The answers are recorded in a standardized form, which includes the following:
Test subject ID
Answer to the question whether test tablets lead to a sweet taste during the time in the mouth (Yes/No)
   d. Evaluation:
The data is statistically analyzed to determine whether a significant number of tester subjects perceived sweetness in the test tablets during the time in the mouth.
   e. Reporting:
The results are summarized in a final report containing the following information:
Number of test subjects who perceived sweetness in the test tablets during the time in the mouth
Statistical analysis of the results
Conclusions regarding the sweetness of the tested tablets
   f. Safety and Ethical Considerations:
All test subjects are informed about possible risks and give their written consent.
The test is carried out under supervision to ensure compliance with the protocols and to provide support if required.
   g. Results

When implementing the Sweet Presence Binary Test for the compositions of examples 1-6, more than 90% of the test subjects, and in the case of examples 2 (n=100) and 3 (n=50) even more than 95% of the test subjects, stated that they did not perceive any sweet taste.

1. A composition in tablet form, consisting of tablet cores containing 70-95% dextrose, lactose, maltose, trehalose, sucrose, maltodextrin or mixtures thereof together with agents customary in the art for improving processability and dissolution properties, respectively and a
tablet coating selected from the following substance classes
a) cellulose and cellulose derivatives, in particular carboxymethyl cellulose, hydroxypropyl methyl cellulose phthalate, methyl cellulose, hydroxypropyl methyl cellulose (hypromellose) and/or hydroxypropyl cellulose,
b) methacrylic acid copolymers and methacrylic acid esters
c) polyvinyl pyrrolidone (PVP) and derivatives
d) polyvidone acetate
e) Polyvinyl acetate phthalate
wherein the tablets obtained from the tablet cores the coating have a diameter of between 4 and 10 mm and a mass of between 50 and 500 mg,
wherein
the tablets do not release carbohydrates into the mouth within 15 seconds and that
after swallowing, the tablets release more than 80% of the carbohydrates contained into the stomach within one minute.

2. The composition in tablet form of claim 1, wherein the individual tablets have a diameter of 7 to 8 mm and a mass of 210 to 270 mg.

3. The composition in tablet form of claim 1, wherein the tablet cores contain about 80% dextrose, lactose, maltose, trehalose, sucrose, maltodextrin or mixtures thereof.

4. The composition in tablet form of claim 1, wherein the tablet cores contain maltodextrin.

5. The composition in tablet form of claim 1, wherein the tablet coating essentially contains hydroxypropyl methyl cellulose.

6. A method comprising:
providing a composition in tablet form for the preparation of an agent for the treatment of hypoglycemic conditions, the composition consisting of tablet cores containing 70-95% dextrose, lactose, maltose, trehalose, sucrose maltodextrin or mixtures thereof together with agents customary in the art for improving processability and dissolution properties, respectively,
and a
tablet coating selected from the following substance classes
a) cellulose and cellulose derivatives, in particular carboxymethyl cellulose, hydroxypropy methyl cellulose phthalate, methyl cellulose, hydroxypropyl methyl cellulose (hypromellose) and/or hydroxypropyl cellulose,
b) methacrylic acid copolymers and methacrylic acid esters
c) polyvinyl pyrrolidone (PVP) and derivatives
d) polyvidone acetate
e) Polyvinyl acetate phthalate
wherein the tablets obtained from the table cores the coating have a diameter of between 4 and 10 mm and a mass of between 50 and 500 mg, and
where the tablets do not release carbohydrates into the mouth within 15 seconds, and
wherein, after swallowing, the tablets release more than 80% of the carbohydrates contained into the stomach within one minute.

7. The method of claim 6, wherein the individual tablets have a diameter of 7 to 8 mm and a mass of 210 to 270 mg.

8. The method of claim 6, wherein the tablet cores contain about 80% dextrose, lactose, maltose, trehalose, sucrose, maltodextrin or mixtures thereof.

9. The method of claim 6, wherein the tablet cores contain maltodextrin.

10. The use of the composition for the treatment of hypoglycemia in tablet form of claim 6, wherein the tablet cores contain about 80% dextrose.

11. The use of the composition for the treatment of hypoglycemia in tablet form of claim 6, wherein the tablet coating essentially contains hydroxypropyl methyl cellulose.
